(19) United States
(12) Patent Application Publication
(10) Pub. No.: US 2012/0290047 A1
(43) Pub. Date: Nov. 15, 2012

(54) SYSTEM AND METHOD FOR DELIVERING LASER ENERGY TO THE BODY

(76) Inventors: John Hendy, Grants Pass, OR (US); Mark D. Wieczorek, San Diego, CA (US)

(21) Appl. No.: 13/424,516

(22) Filed: Mar. 20, 2012

Related U.S. Application Data
(60) Provisional application No. 61/484,921, filed on May 11, 2011.

(51) Int. Cl. A61N 5/067; A61N 5/06

(52) U.S. Cl. 607/89; 607/92

(57) ABSTRACT

Systems and methods are provided that allow treatment of cells that are otherwise inaccessible to low level laser therapy. An optically-transmissive device, e.g., a clear piece of silicone, may be placed within a patient's body to optically connect a surface portion, where a laser may be located, to a deeper portion, where a tissue bed to be treated may be located.

### CROSS-REFERENCE TO RELATED APPLICATIONS

This application claims benefit of priority of U.S. Provisional Patent Application Ser. No. 61/484,921, filed May 11, 2011, entitled “SYSTEM AND METHOD FOR DELIVERING LASER ENERGY TO THE INACCESSIBLE AREAS OF THE BODY”, incorporated by reference herein in its entirety.

### BACKGROUND

Lasers have been employed to treat a number of medical indications. In many cases, the same are employed to destroy or necrose tissue or other anatomical parts, including the use of lasers in dentistry. Beyond usage to destroy or necrose tissue, laser impingement on surface cells has been used to therapeutically treat the cells, where the laser power is at an appropriately low level. However, where cells are not easily accessible, laser treatments are unavailable.

### SUMMARY

Systems and methods disclosed here allow treatment of cells that are otherwise inaccessible to low level laser therapy. An optically-transmissive device, e.g., a clear piece of silicone, may be placed within a patient's body to optically connect a surface portion, where a laser may be located, to a deeper portion, where a tissue bed to be treated may be located.

In one aspect, the invention is directed towards a device for treatment of tissue, including: a shaft made of a light transmissive material, the shaft including a distal end and a proximal end, the distal end of the shaft configured to be inserted into tissue of a patient and the proximal end of the shaft configured to be mounted intradermally adjacent a light source; and the shaft including a light transmissive feature at least at the distal end, the light transmissive configured to emit light into tissue.

Implementations of the invention may include one or more of the following. The device may further include a shoulder at a proximal end of the shaft, the shoulder for mounting the shaft adjacent the light source. The light source may be a laser or a light emitting diode. The light transmissive feature may unit light in an azimuthally-symmetric direction or in a non-azimuthally-symmetric direction. The light transmissive feature may include at least one irregularity, such as one or more dimples or bumps or divets. The light transmissive feature may include a round end, a flat end, or a geodesic feature, or the like. The light transmissive feature may run substantially the length of the device, or may be situated at a distal end, such as at the last two inches from the distal tip. The light source may provide light in an energy range of from 1 mJ to 10 J, e.g., from 1 mJ to 4 or 5 J, e.g., in the mJ range or low single digit J range. The shaft may be elongated. The shaft and shoulder may form a plug.

In another aspect, the invention is directed towards a method of treating tissue, including: inserting a device into a patient, the device including a shaft made of a light transmissive material, the shaft including a distal end and a proximal end, the distal end of the shaft configured to be inserted into tissue of a patient and the proximal end of the shaft configured to be mounted adjacent a light source; and the shaft including a light transmissive feature at least at the distal end, the light transmissive configured to emit light into tissue. The proximal end is then coupled to a light source, and the light source is activated to provide light in an energy range of from 1 mJ to 10 J, e.g., from 1 mJ to 4 J.

Implementations of the invention may include one or more of the following. The inserting may be performed internally or externally. The method may be for the treatment of the brain, the liver, the skin, the esophagus, the colon, the lung, the kidney, the pancreas, the intestinal tract, blood vessels, or the chest.

Advantages include that cells that are otherwise inaccessible may be reachable and subject to treatment. Other advantages will be apparent from the description that follows, including the figures and claims.

### BRIEF DESCRIPTION OF THE DRAWINGS

FIG. 1A illustrates a side view of a first embodiment according to the principles described here.

FIG. 1B illustrates a top view of the embodiment of FIG. 1A.

FIG. 1C illustrates a system employing certain embodiments according to the principles described here.

FIGS. 1(D)-1(E) illustrate different types of distal tips, according to the principles described here.

FIGS. 2(A)-2(C) illustrate different types of elongated embodiments according to the principles described here.

Like reference numerals reference like elements throughout.

### DETAILED DESCRIPTION

The present invention allows treatment of cells that are otherwise inaccessible to low level laser therapy. An optically-transmissive device, e.g., a clear piece of silicone, may be placed within a patient's body to optically connect a surface portion, where a laser may be located, to a deeper portion, where a tissue bed to be treated may be located. Other types of optically transmissive devices may also be employed, including: acrylics (either soft or hard), as well as other materials, e.g., such as silicone bags filled with water-based constituents, e.g., materials similar to breast implants. Such latter materials may be useful where the device is implanted for periodic treatments over an extended time, as the same would not cause damage if struck.

The devices as noted may be buried below the skin for extended periods of time for long term cases, e.g., diabetes, or may be employed for short-term use.

In general, the devices provided may be short or long or any length in between. In a longer embodiment, the device may include an elongated flexible or rigid member to provide an optical connection between a laser and a tissue bed to be treated. In situations where the tissue bed to be treated cannot be accessed directly by the beam of a laser, the device may be particularly advantageously employed.

In one exemplary implementation, a short rigid device may be employed that is inserted into a patient's skull to deliver laser energy to the brain, and in particular to tissue affected by stroke, trauma, or other deleterious and especially ischemic influences. It is known to remove a small plug from a patient's skull to ease pressure within the same, and by accessing such an area, the device may be employed to transmit laser energy.

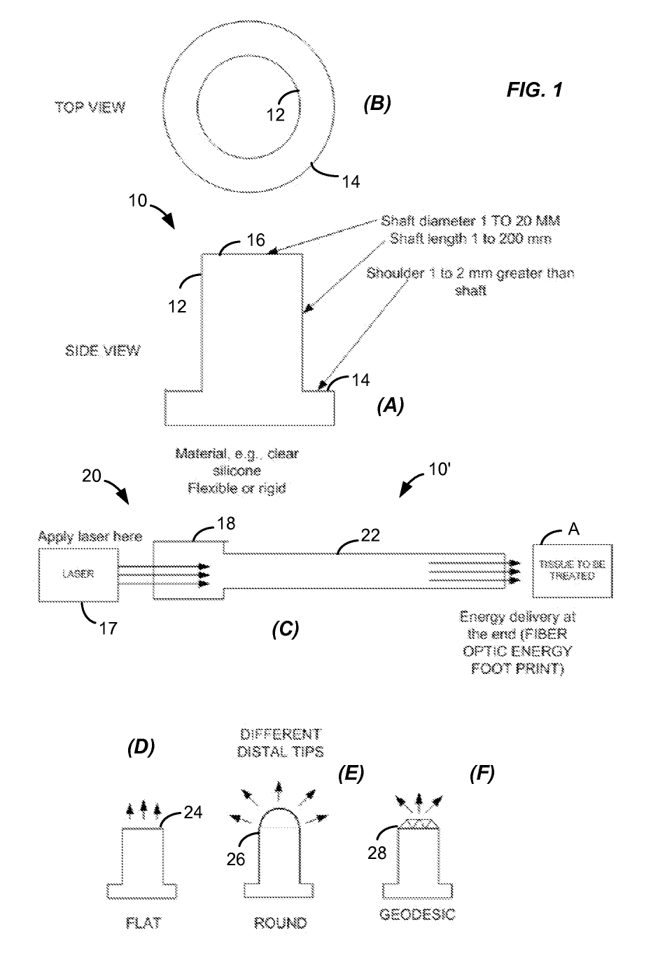

Such a device is shown in FIG. 1. In FIG. 1A, a short device 10 is illustrated in side view, having a shaft 12, a shoulder 14, and a distal tip 16. Various dimensions are given for the shaft diameter and length, as well as the shoulder. FIG. 1B illustrates the device in top view.

FIG. 1C illustrates device 10′ in use in a system 20. The shaft has been illustrated as an elongated shaft 22, which is coupled at a proximal end to a shoulder 18. A laser 17 illuminates the system, which causes tissue A to be treated.

Various types of distal tips may be employed, according to the type of laser treatment necessary. Different distal tips are shown in FIGS. 1(D)-1(F), including a flat distal tip 24, a round distal tip 26, and a geodesic distal tip 28. It will be understood that other types of distal tips may also be employed, as may be required for the desired type of laser treatment.

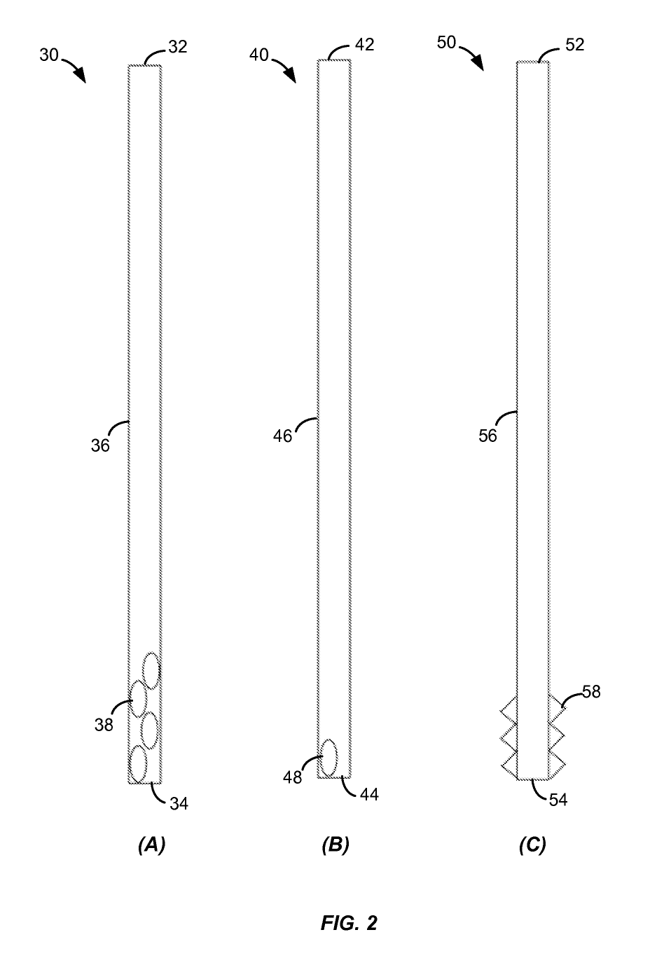

In another exemplary implementation, a long flexible device may be inserted into a patient's abdomen to deliver laser energy to deep tissues. For example, referring to FIGS. 2(A)-2(C), various embodiments are illustrated of elongated devices. In particular, devices, 30, 40, and 50 are illustrated with proximal ends 32, 42, and 52, distal ends 34, 44, and 54, and shafts 36, 46, and 56, respectively.

The devices of FIGS. 2(A)-2(C) are illustrated with various types of laser light-transmissive ends. In FIG. 2A, a series of dimples 38 are provided which generally transmit light in azimuthally-symmetric directions. The dimples 38 may be generally similar to what is on a golf ball. In another fashion, instead of dimples, small bumps extending out of the shaft 36 may be provided.

In FIG. 2B, a single dimple (or bump) 48 is illustrated, the same providing laser light treatment in a non-azimuthally-symmetric direction. In particular, the same would generally emit light in a single direction, allowing for particularly carefully directed laser therapy.

In FIG. 2C, a series of divets 58 are illustrated, the same providing laser light treatment in azimuthally-symmetric directions, but in a different pattern than that provided by the device of FIG. 2A. In a related implementation, the divets may increase in size the farther one goes down the shaft. In other words, divets at the distal end may be larger than those closer to the proximal end. In this way, a more even distribution of laser light is provided, as the light at the distal end may become more attenuated, due to scattering, absorption, and extinction within the shaft.

It will be understood that other types of laser transmissive features may also be provided. In such systems as are shown in FIG. 2, the laser transmissive features may be provided over a significant part of the shaft or may be localized at the distal end, e.g., at the last 1-2″. Where the same are localized, the physician may be provided with a greater degree of control over where the laser treatment is being applied. The type of light delivery can be changed by the employment of different distal tips.

The system and method may be employed in a number of indications. For example, the device may be inserted internally, e.g., in the brain, the system and method may be employed in the treatment of traumatic brain injury, stroke rehabilitation, depression, and healing after surgery. Also in the brain, the system and method may be employed in the treatment of migraine headaches, or to improve cell health, particularly blood vessel cells, to help prevent stroke.

In abdominal indications, where again the device is inserted internally, the system and method may be employed to assist in the healing of injuries due to surgery. The system and method may be employed in the pancreas, such as in the treatment of diabetes. The system and method may be employed for the regeneration of liver tissue for the treatment of cirrhosis. The system and method may be employed to treat lung tissue to help such tissue after smoke inhalation or other damage. The system and method may be employed to treat kidneys, such as to regenerate damaged tissue after the ingestion of drugs or poisons. The system and method may be employed to treat injuries to the intestinal tract or to a crushed chest. The system and method may be employed to treat the colon, such as in a rectal approach for healing damage of colitis or surgery. The system and method may be employed on the skin to treat bruising.

In another exemplary implementation, the device may be inserted externally, e.g., fed through the nose or mouth, depending on the patient's gag reflex, down the esophagus for treatment of acid reflux damage or deeper for Gastritis. A “barium swallow” may be employed to determine the length of the esophagus, and thus the size of the device needed. In such a system, the device may be inserted all the way down to the bottom of the esophagus or into the stomach, and then slowly brought back up while performing the laser treatment. This method can also be used to insert into the vagina and uterus to treat endometriosis.

In certain implementations, the device and system may be employed not only with low level laser therapy but also in combination with stem cell treatments.

While the amount of energy delivered to the tissue will vary greatly, in some cases 1 mJ to 10 J of energy delivered to the tissue has been found appropriate, e.g., 1 mJ-5 J of energy, or, e.g., 1 mJ-4 J of energy, such as 3 J of energy. In some instances, lasers operating in the wavelength of 810-830 nm have been employed, although it will be understood that other types of lasers may also be used. It will be understood that various peripheral devices known to work with lasers may also be employed in systems and methods according to the principles described here. For example, “elbow” fiber bundles for transmitting laser light around curves may be employed, and the like.

In addition, while various types of light transmissive features on distal tips have been disclosed, one of skill in the art will recognize given this teaching that such are dependent on application. Depending on application, generalized irregularities may be employed, including those created by scratching, cutting, sandblasting, and the like. In some embodiments, the light transmissive feature may be simply the end of the tip, e.g., the distal end of the cylindrical (or other volumetric) shaft.

While laser treatments have been disclosed here, it will be understood that in this type of treatment, it is the light itself that affects the tissue, not the coherency of the light. Accordingly, an LED may also be employed. In the same way, a super luminous diode or other light source may be used.

Various illustrative implementations of the present invention have been described. However, one of ordinary skill in the art will recognize that additional implementations are also possible and within the scope of the present invention. Accordingly, the invention is to be limited only by the claims appended hereto.

1. A device for treatment of tissue, comprising:
a. a shaft made of a light transmissive material, the shaft including a distal end and a proximal end, the distal end of the shaft configured to be inserted into tissue of a patient and the proximal end of the shaft configured to be mounted in a subdermal location adjacent a light source; and
b. the shaft including a light transmissive feature at least at the distal end, the light transmissive configured to emit light into tissue.

2. The device of claim 1, further comprising a shoulder at a proximal end of the shaft, the shoulder for mounting the shaft adjacent the light source.

3. The device of claim 2, wherein the light source is a laser.

4. The device of claim 2, wherein the light source is a light emitting diode.

5. The device of claim 1, wherein the light transmissive feature emits light in an azimuthally-symmetric direction.

6. The device of claim 1, wherein the light transmissive feature emits light in a non-azimuthally-symmetric direction.

7. The device of claim 1, wherein the light transmissive feature includes at least one irregularity.

8. The device of claim 7, wherein the irregularity includes one or more dimples or bumps.

9. The device of claim 7, wherein the irregularity includes one or more divets.

10. The device of claim 1, wherein the light transmissive feature includes a round end.

11. The device of claim 1, wherein the light transmissive feature includes a flat end.

12. The device of claim 1, wherein the light transmissive feature includes a geodesic feature.

13. The device of claim 1, wherein the light transmissive feature is only at the last two inches from the distal end.

14. The device of claim 1, wherein the light source provides light in an energy range of from 1-10 J.

15. The device of claim 14, wherein the light source provides light in an energy range of from 2-5 J.

16. The device of claim 15, wherein the light source provides light in an energy range of from 2-4 J.

17. The device of claim 1, wherein the shaft and shoulder form a plug.

18. A method of treating tissue, comprising:
a. inserting the device of claim 1 into a patient;
b. coupling the proximal end to a light source; and
c. activating the light source to provide light in an energy range of from 1 mJ-10 J.

19. The method of claim 18, wherein the inserting includes inserting internally or externally.

20. The method of claim 18, wherein the treatment is for the treatment of the brain, the liver, the skin, the esophagus, the colon, the lung, the kidney, the pancreas, the intestinal tract, blood vessels, vagina, uterus or the chest.
